(19) United States
(12) Patent Application Publication
(10) Pub. No.: US 2021/0389290 A1
(43) Pub. Date: Dec. 16, 2021

(54) Intelligent Monitoring and Analysis Method for Air Pollution and Device Thereof

(71) Applicant: CHINESE RESEARCH ACADEMY OF ENVIRONMENTAL SCIENCES, Beijing (CN)

(72) Inventors: LIEYU ZHANG, Beijing (CN); Gengyuan Liu, Beijing (CN); Guowen Li, Beijing (CN); Lulu Che, Beijing (CN); Xiaoguang LI, Beijing (CN); Jiaqian Li, Beijing (CN); Chen Zhao, Beijing (CN); WEI LI, BEIJING (CN)

(21) Appl. No.: 17/242,523

(22) Filed: Apr. 28, 2021

(30) Foreign Application Priority Data
Jun. 12, 2020 (CN) 2020105383513

(51) Int. Cl. G01N 33/00

(52) U.S. Cl. CPC G01N 33/0062

(57) ABSTRACT

Embodiments of the present application relate to methods, devices, apparatuses, and storage media for monitoring and analyzing air pollution. The methods includes acquiring air quality data of a monitored area, where the air quality data includes ground air quality data corresponding to the monitored area and N-component data collected by an air sensor in the monitored area; inputting the air quality data into a pollution analysis model, where the pollution analysis model is previously trained according to the ground air quality data and the N-component data; and determining whether the air quality data of the monitored area is abnormal according to an output result of the pollution analysis model. The methods may solve the technical problem of inaccurate monitoring results caused by the susceptibility of air quality monitoring in related technologies to human intervention.

### CROSS REFERENCE TO RELATED APPLICATION

This application claims the benefit of Chinese Patent Application No. 2020105383513, filed Jun. 12, 2020, which is hereby incorporated by reference herein in its entirety.

### TECHNICAL FIELD

The present application relates to the field of air pollution monitoring, in particular to methods, devices, apparatuses, and storage media for monitoring and analyzing air pollution air pollution.

### BACKGROUND

The air pollution index has been increasingly important in various places. In addition to normal ways of environmental governance, some areas have also “falsified” data of air quality monitoring stations. There are measures such as covering air samplers with cotton wool or masks, blocking large vehicles for cleaning near monitoring stations, and performing artificial spraying on monitoring points using spray cannons to purify the air. These behaviors have caused large errors in the data of more than 1400 air quality monitoring state-controlled points, undermining the reliability of the overall system. However, since these behaviors rarely damage the air monitoring station entity and the data generation process is normal, it is difficult to fully and effectively discover such falsification behaviors, and 24-hour real-time manual monitoring of the surrounding environment is too costly and difficult to implement.

The aerosol optical thickness measurement method, the trace gas quantitative remote sensing method and the like used by the environmental protection satellite can effectively analyze the air quality near the ground and make quantitative judgment of types of pollutants (such as haze, polluting gases, greenhouse gases and the like) to some degree. The aerosol optical thickness measurement method measures the value of aerosol optical thickness. The aerosol optical thickness is defined as the integral of the extinction coefficient of the medium in the vertical direction, which describes the reduction effect of aerosol on light. The aerosol optical thickness characterizes the degree of atmospheric turbidity. The value of aerosol optical thickness predicts the growth of aerosol accumulation in the longitudinal direction, which leads to the decrease of atmospheric visibility. The higher the value of aerosol optical thickness, the lower the visibility, and the more serious the air pollution. Due to the wide coverage of satellite monitoring, the difficulty and cost of data falsification is extremely high. The source of data is single, so it can be considered currently as the environmental monitoring method with the highest confidence.

However, the main problem of satellite monitoring is that due to the continuous movement of the satellite and the change of the scanning trajectory, continuous data monitoring of all areas cannot be performed, so the data of a specific area is in a non-continuous monitoring state. The data of air quality monitoring state-controlled points is prone to “artificial” errors locally, but the data of air quality monitoring state-controlled points is continuous and the density of collected data is high.

For the above problems, no effective solutions have been proposed yet.

### SUMMARY

Embodiments of the present application provide methods, devices, apparatuses, systems, and computer media for monitoring and analyzing air pollution to at least solve the technical problem of inaccurate monitoring results caused by the susceptibility of air quality monitoring in related technologies to human intervention.

According to one aspect of the embodiments of the present application, a method for monitoring and analyzing air pollution is provided. The method includes the following steps of: acquiring air quality data of a monitored area, where the air quality data includes ground air quality data corresponding to the monitored area and N-component data collected by an air sensor in the monitored area, where N is a positive integer; inputting the air quality data into a pollution analysis model, wherein the pollution analysis model is previously trained according to the ground air quality data and the N-component data; and determining whether the air quality data of the monitored area is abnormal according to an output result of the pollution analysis model.

Further, the method includes training the pollution analysis model according to air quality training data, which may include: collecting ground air quality sample data and N-component sample data corresponding to a sample area at a preset sampling time interval; determining an air quality label of the sample area according to the air quality sample data; and constructing an air quality data set with the air quality label and the N-component sample data according to sampling time.

Further, the step of determining the air quality label of the sample area according to the air quality sample data includes acquiring an aerosol optical thickness and trace gas quantitative remote sensing parameters in the air quality sample data; and quantitatively determining the corresponding air quality label according to the aerosol optical thickness and trace gas quantitative remote sensing parameters.

Further, the step of constructing the air quality data set with the air quality label and the N-component sample data according to the sampling time includes performing dimensionality reduction on the N-component sample data corresponding to the air quality label according to a principal component analysis method to obtain the air quality data set.

Further, the method further includes: after constructing the air quality data set with the air quality label and the N-component sample data according to the sampling time, performing one or more operations. The operations include: dividing the air quality data set collected within a preset time period into training samples and test samples; testing model parameters in the air pollution analysis model according to the training samples; and verifying the accuracy of the air pollution analysis model according to the test samples.

Further, the step of determining whether the air quality data of the monitored area is abnormal according to the output result of the pollution analysis model includes: in response to determining that the air quality data of the monitored area is abnormal, comparing a sampling duration in the N-component data with a corresponding preset abnormality duration threshold, respectively; and in some embodiments in which the sampling duration of the component data is greater than the preset abnormality duration threshold, determining that the component data is abnormal data.

The method further includes: after determining that the component data is the abnormal data, in the case where it is determined that the component data is the abnormal data, acquiring the sampling time of the abnormal data, and acquiring position information of the sensor for collecting the abnormal data; and analyzing the N-component data in the time period during which the sampling time is located.

According to another aspect of the embodiments of the present application, an apparatus for monitoring and analyzing air pollution is provided. The apparatus includes: an acquiring unit, configured to acquire air quality data of a monitored area, where the air quality data includes ground air quality data corresponding to the monitored area and N-component data collected by an air sensor in the monitored area, where N is a positive integer; a processing unit, configured to input the air quality data into a pollution analysis model, where the pollution analysis model is previously trained according to the ground air quality data and the N-component data; and a determining unit, configured to determine whether the air quality data of the monitored area is abnormal according to an output result of the pollution analysis model.

According to another aspect of the embodiments of the present application, a storage medium is provided. The storage medium includes a stored program, where when the program is running, the intelligent monitoring and analysis method for air pollution as described above is executed.

According to another aspect of the embodiments of the present application, an electronic device is provided. The electronic device includes a memory, a processor and a computer program stored in the memory and capable of running on the processor, where the processor executes the intelligent monitoring and analysis method for air pollution as described above by the computer program.

In the embodiments of the present application, air quality data of a monitored area is acquired, where the air quality data includes ground air quality data corresponding to the monitored area and N-component data collected by an air sensor in the monitored area; the air quality data is input into a pollution analysis model, where the pollution analysis model is previously trained according to the ground air quality data and the N-component data; and whether the air quality data of the monitored area is abnormal is judged according to an output result of the pollution analysis model. The objective of combining the ground air quality data collected by a satellite and the component data collected by the ground air sensor in the monitored area is achieved, thereby realizing the technical effect of more accurate air quality monitoring results. Thereby, the method solves the technical problem of inaccurate monitoring results caused by the susceptibility of air quality monitoring in related technologies to human intervention.

### BRIEF DESCRIPTION OF THE DRAWINGS

In order to more clearly illustrate the technical solutions of embodiments of the present application, the accompanying drawings that need to be used in the description of the embodiments or the prior art will be briefly described below. Obviously, the accompanying drawings in the following description are only some embodiments of the present application, and those of ordinary skill in the art can obtain other accompanying drawings according to these accompanying drawings without any creative effort.

FIG. 1 is a schematic diagram of an optional air pollution intelligent monitoring and analysis method according to an embodiment of the present application; and

FIG. 2 is a schematic diagram of an optional air pollution intelligent monitoring and analysis device according to an embodiment of the present application.

### DETAILED DESCRIPTION

In order to make the objectives, technical solutions and advantages of the embodiments of the present application clearer, the technical solutions in the embodiments of the present application will be clearly and completely described below in conjunction the accompanying drawings in the embodiments of the present application. It is apparent that the described embodiments are a part of the embodiments of the present application, rather than all of the embodiments. All other embodiments obtained by those of ordinary skill in the art based on the embodiments in the present application without creative efforts shall fall within the protection scope of the present application.

It should be noted that relational terms such as “first” and “second” herein are only used to distinguish one entity or operation from another entity or operation, and do not necessarily require or imply any such actual relationship or sequence between these entities or operations.

### Embodiment 1

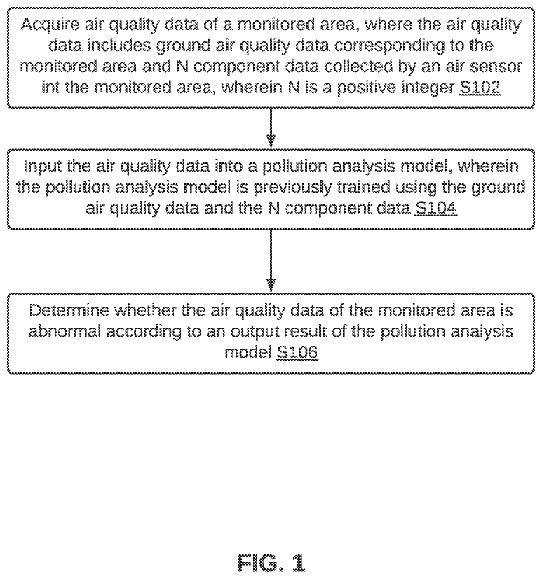

According to the embodiment of the present application, a method for monitoring and analyzing air pollution is provided. As shown in FIG. 1, the method includes: S102, acquiring air quality data of a monitored area, where the air quality data includes ground air quality data corresponding to the monitored area and N-component data collected by an air sensor in the monitored area, where N is a positive integer;

S104, inputting the air quality data into a pollution analysis model, where the pollution analysis model is previously trained according to the ground air quality data and the N-component data; and

S106, determining whether the air quality data of the monitored area is abnormal according to an output result of the pollution analysis model.

In this embodiment, by acquiring the ground air quality data collected by the satellite and the multiple component data collected by the ground air sensor in the monitored area, the air quality of the monitored area can be monitored and analyzed from multiple dimensions in the air and ground. The component data is generally pollutant components, such as BrO, NOₓ, CH₄ and the like. Specifically, the air quality data generally includes a set of materials such as pictures or videos taken by the satellite and N pollutant components collected by the ground air sensor, and the types of specific pollutant components can be set based on actual experience.

In a specific application scenario, the air quality data in the sample area, that is, ground air quality data and N-component data, is acquired. The air quality data in the sample area needs to include air quality in various situations, and the pollution analysis model generally adopts a support vector machine (SVM) model.

It should be noted that in this embodiment, whether the air quality data of the monitored area is abnormal is determined by acquiring the air quality data of the monitored area and inputting the air quality data into the previously trained pollution analysis model to realize the identification of human intervention in air quality monitoring.

Alternatively, in this embodiment, the pollution analysis model is previously trained using air quality training data. The pollution analysis model may collect ground air quality sample data and N-component sample data corresponding to a sample area at a preset interval; determine an air quality label of the sample area according to the air quality sample data; and construct an air quality data set with the air quality label and the N-component sample data according to sampling time.

In an actual application scenario, in the process of monitoring the air in a certain area, the air quality data of the monitored area is collected every preset time. Therefore, in this embodiment, in the training process of the pollution analysis model, the ground air quality sample data collected by the satellite corresponding to the sample area and the N-component sample data collected by the ground air sensor are collected at a preset sampling time interval. Then the air quality sample data and the N-component sample data are filtered to obtain filtered movement data. The air quality of the sample area is determined according to the ground air quality sample data collected by the satellite. This air quality is the pollution status of the sample area. The air quality label of the sample area is determined according to the air quality, then the N-component sample data collected at the same sampling time is labeled, and accordingly, the air quality data set is constructed according to the air quality sample data and the N-component sample data collected within the specified time period.

Alternatively, in this embodiment, determining the air quality label of the monitored area according to the air quality sample data includes but is not limited to: acquiring an aerosol optical thickness and trace gas quantitative remote sensing parameters in the air quality sample data; and quantitatively determining the corresponding air quality label according to the aerosol optical thickness and trace gas quantitative remote sensing parameters.

In a specific application scenario, the air quality of the sample area is determined by an aerosol optical thickness measurement method and a trace gas quantitative remote sensing method used by the environmental protection satellite. For example, vertical aerosol distribution data is acquired from a meteorological satellite, entire aerosol data distribution is simulated by using a radiative transfer model, and elevation data distribution of the aerosol vertical distribution is acquired by combining the observed ground extinction coefficient; and the obtained elevation data distribution is subjected to humidity correction, and the ground aerosol extinction coefficient is decomposed from the entire aerosol data distribution.

Alternatively, in this embodiment, constructing the air quality data set with the air quality label and the N-component sample data according to the sampling time includes but is not limited to: performing dimensionality reduction on the N-component sample data corresponding to the air quality label according to a principal component analysis method to obtain the air quality data set.

Specifically, according to the types of the pollutant components presented in the air quality data, a preset number of sample data is selected for each type of component to form eigenvectors of the training model as condition attributes for determining air quality. According to the sampling time and the air quality label corresponding to the air quality data, the dimensionality of the eigenvectors is reduced by the principal component analysis method. The dimensionality-reduced data eigenvectors may be used to form the air quality data set. In an actual application process, multiple eigenvalues in the eigenvectors that reflect the data information are compressed into several principal components, where each principal component can reflect most of the information of the original eigenvector, and the information contained is not repeated.

Alternatively, in this embodiment, after constructing the air quality data set with the air quality label and the N-component sample data according to the sampling time, the method further includes, but is not limited to: dividing the air quality data set collected within a preset time period into training samples and test samples; testing model parameters in the air pollution analysis model according to the training samples; and verifying the accuracy of the air pollution analysis model according to the test samples.

In a specific application scenario, the air quality data is divided into the training samples and the test samples, the SVM model is constructed according to the training samples and the test samples. The training samples are used to test the penalty coefficient, kernel function and other parameters in the SVM model, and the test samples are used to verify the accuracy of the model.

Specifically, in the sample extraction method and the construction of the air quality data set, several continuous data points are extracted for each component sample data by using time as the axis, corresponding x data of m continuous data points for each type of data is taken to count the eigenvalues. There are N*x data eigenvalues in total for N types of data. Then the eigenvalues are subjected to principal component analysis and compressed into several principal components, which are used together with a new eigenvalue vector formed by a falsified value as a sample. The sample sampling interval is the sampling time of d continuous data points, which is d*0.1 s. Multiple component samples collected continuously form a sample set. m, t and d are all positive integers.

Alternatively, in this embodiment, the determining whether the air quality data of the monitored area is abnormal according to the output result of the pollution analysis model includes, but is not limited to: in a case where the air quality data of the monitored area is abnormal, respectively comparing a sampling duration in the N-component data with a corresponding preset abnormality duration threshold; and in a case where the sampling duration of the component data is greater than the preset abnormality threshold, determining that the component data is abnormal data.

In a specific application scenario, the characteristic parameters of the trained pollution analysis model are transferred to an edge computing gateway module in the monitored area. The edge computing gateway module can perform low-power-consumption high-performance computing. Through the computing of the edge computing gateway module and the real-time classification of sensor data of the mobile phone, it is judged whether there is abnormal data. In the case where the air quality data of the monitored area is abnormal, the component data whose initial judgment result is abnormal is subjected to judgment a second time. The value corresponding to the sampling time of the component data is compared with the abnormality threshold, and in the case where the falsified value is greater than the abnormality threshold, it is judged that the component data is the abnormal data.

Alternatively, in this embodiment, after determining that the component data is the abnormal data, the method further includes, but is not limited to: in the embodiments in which it is determined that the component data is the abnormal data, acquiring the sampling time of the abnormal data, and acquiring position information of the sensor for collecting the abnormal data; and analyzing the N-component data in the time period during which the sampling time is located.

Specifically, after the abnormal data is identified, analysis is performed based on the N-component data adjacent to the abnormal data in time series. The position information of the sensor for collecting the abnormal data is acquired.

The air quality data of the monitored area is acquired, where the air quality data includes ground air quality data corresponding to the monitored area and N-component data collected by an air sensor in the monitored area; the air quality data is input into a pollution analysis model, where the pollution analysis model is previously trained according to the ground air quality data and the N-component data; and whether the air quality data of the monitored area is abnormal is judged according to the output result of the pollution analysis model. The objective of combining the ground air quality data collected by the satellite and the component data collected by the ground air sensors in the monitored area is achieved, thereby realizing the technical effect of more accurate air quality monitoring results. Thereby, the method solves the technical problem of inaccurate monitoring results caused by the susceptibility of air quality monitoring in related technologies to human intervention.

It should be noted that for the foregoing method embodiments, for the sake of simple description, they are all expressed as a combination of a series of actions, but those skilled in the art should know that the present application is not limited by the described sequence of actions, because according to the present application, some steps can be performed in other sequence or simultaneously. Secondly, those skilled in the art should also know that the embodiments described in the specification are all preferred embodiments, and the actions and modules involved are not necessarily required by the present application.

Through the description of the above implementations, those skilled in the art can clearly understand that the method according to the above embodiments can be implemented by means of software plus a necessary general hardware platform, and of course, it can also be implemented by hardware, but in many cases the former is a better implementation. Based on such an understanding, the technical solution of the present application essentially or for the part that contributes to the prior art can be embodied in the form of a software product, and the computer software product is stored in a storage medium (such as a ROM/RAM, a magnetic disk, an optical disk) and includes several instructions to enable a terminal facility (which may be a mobile phone, a computer, a server, a network facility or the like) to execute the method described in the embodiments of the present application.

### Embodiment 2

According to the embodiment of the present application, an apparatus for monitoring and analyzing intelligent air pollution is provided. The apparatus may implement the method for monitoring and analyzing air pollution described herein. As shown in

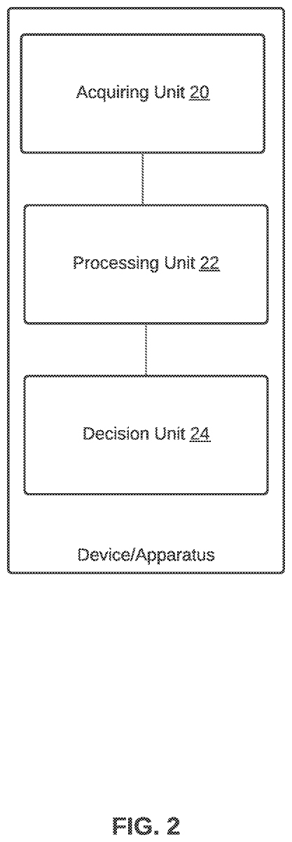

FIG. 2, the apparatus may include:

1) an acquiring unit 20 configured to acquire air quality data of a monitored area, where the air quality data includes ground air quality data corresponding to the monitored area and N-component data collected by an air sensor in the monitored area, where N is a positive integer;

2) a processing unit 22 configured to input the air quality data into a pollution analysis model, where the pollution analysis model is previously trained according to the ground air quality data and the N-component data; and

3) a decision unit 24 configured to determine whether the air quality data of the monitored area is abnormal according to an output result of the pollution analysis model.

Alternatively, for the specific example in this embodiment, reference may be made to the example described in Embodiment 1 above, and detailed descriptions will not be repeated here in this embodiment.

### Embodiment 3

According to the embodiment of the present application, a storage medium is further provided, where the storage medium includes a stored program, where when the program is running, the intelligent monitoring and analysis method for air pollution as described above is executed.

Alternatively, in this embodiment, the storage medium is configured to store program codes for executing the following steps:

S1, acquiring air quality data of a monitored area, where the air quality data includes ground air quality data corresponding to the monitored area and N-component data collected by an air sensor in the monitored area, where N is a positive integer;

S2, inputting the air quality data into a pollution analysis model, where the pollution analysis model is previously trained according to the ground air quality data and the N-component data; and

S3, determining whether the air quality data of the monitored area is abnormal according to an output result provided by the pollution analysis model.

Alternatively, in this embodiment, the above storage medium may include, but is not limited to a USB flash disk, a read-only memory (ROM), a random-access memory (RAM), a mobile hard disk, a magnetic disk, an optical disk, or any medium that can store program codes.

Alternatively, for the specific example in this embodiment, reference may be made to the example described in Embodiment 1 above, and detailed descriptions will not be repeated here in this embodiment.

### Embodiment 4

The embodiment of the present application further provides an electronic device, including a memory, a processor and a computer program stored in the memory and capable of running on the processor, where the processor executes the intelligent monitoring and analysis method for air pollution as described above by the computer program.

Alternatively, in this embodiment, the memory is configured to store program codes for executing the following steps:

S1, acquiring air quality data of a monitored area, where the air quality data includes ground air quality data corresponding to the monitored area and N-component data collected by an air sensor in the monitored area, where N is a positive integer;

S2, inputting the air quality data into a pollution analysis model, where the pollution analysis model is previously trained according to the ground air quality data and the N-component data; and

S3, determining whether the air quality data of the monitored area is abnormal according to an output result of the pollution analysis model.

Alternatively, for the specific example in this embodiment, reference may be made to the example described in Embodiment 1 above, and detailed descriptions will not be repeated here in this embodiment.

The serial numbers of the embodiments of the present application above are merely for the description, and do not represent the quality of the embodiments.

When the integrated unit in the embodiments above is implemented in a form of a software function unit and sold or used as an independent product, the integrated unit may be stored in the computer-readable storage medium above. Based on such an understanding, the technical solution of the application essentially or for the part that contributes to the prior art or all or part of the technical solution can be embodied in the form of a software product, and the computer software product is stored in a storage medium and includes several instructions configured to enable one or more computer facilities (which may be a personal computer, a server, a network facility or the like) to execute all or part of the steps of the methods of the embodiments of the present application.

In the above embodiments of the present application, the description for each embodiment has its own focus. For parts that are not described in detail in a certain embodiment, reference may be made to related descriptions of other embodiments.

In the several embodiments provided in the present application, it should be understood that the disclosed client can be implemented in other ways. The device embodiments described above are only schematic. For example, the division of units is only a division of logical functions. In an actual implementation, there may be other division manners, for example, a plurality of units or components may be combined or may be integrated into another system, or some features may be omitted or not executed. In addition, the displayed or discussed mutual coupling or direct coupling or communication connection may be indirect coupling or communication connection through some interfaces, units or modules, and may be in electrical or other forms.

The units described as separate components may or may not be physically separated, and the components displayed as units may or may not be physical units, that is, they may be located in one place, or may be distributed in a plurality of network units. Part or all of the units may be selected according to actual needs to achieve the purposes of the solution of this embodiment.

In addition, the function units in each embodiment of the present application may be integrated into one processing unit, or each unit may exist alone physically, or two or more units may be integrated into one unit. The above integrated unit may be implemented in the form of hardware or implemented in the form of a software function unit.

The above description is only preferred implementations of the present application. It should be noted that those of ordinary skill in the art may also make several improvements and modifications without departing from the principles of the present application, and such improvements and modifications should also be regarded as the protection scope of the present application.

1. A method for monitoring and analyzing air pollution, comprising:
acquiring air quality data of a monitored area, wherein the air quality data comprises ground air quality data corresponding to the monitored area and N-component sample data collected by an air sensor in the monitored area, wherein N is a positive integer;
inputting the air quality data into a pollution analysis model, wherein the pollution analysis model is previously trained according to the ground air quality data and the N-component data; and
determining whether the air quality data of the monitored area is abnormal according to an output result of the pollution analysis model.

2. The method of claim 1, further comprising training the pollution analysis model according to air quality training data by:
collecting the ground air quality sample data corresponding to the monitored area and the N-component sample data corresponding to a sample area at a preset sampling time interval;
determining an air quality label of the sample area according to the air quality sample data; and
constructing an air quality data set with the air quality label and the N-component sample data according to sampling time.

3. The method of claim 2, wherein determining the air quality label of the sample area according to the air quality sample data comprises:
acquiring an aerosol optical thickness and trace gas quantitative remote sensing parameters in the air quality sample data; and
quantitatively determining the air quality label according to the aerosol optical thickness and trace gas quantitative remote sensing parameters.

4. The method of claim 2, wherein constructing the air quality data set with the air quality label and the N-component sample data according to the sampling time comprises:
performing dimensionality reduction on the N-component sample data corresponding to the air quality label according to a principal component analysis method to obtain the air quality data set.

5. The method of claim 4, further comprising after constructing the air quality data set with the air quality label and the N-component sample data according to the sampling time, performing a plurality of operations, wherein the plurality of operations comprises:
dividing the air quality data set collected within a preset time period into training samples and test samples;
testing model parameters in the pollution analysis model according to the training samples; and
verifying whether the pollution analysis model is accurate according to the test samples.

6. The method of claim 1, wherein determining whether the air quality data of the monitored area is abnormal according to the output result of the pollution analysis model comprises:
in response to determining that the air quality data of the monitored area is abnormal, respectively comparing a sampling duration in the N-component data with a corresponding preset abnormality duration threshold; and
in response to determining that the sampling duration of the component data is greater than the preset abnormality duration threshold, determining that the component data is abnormal data.

7. The method of claim 6, further comprising performing a plurality of operations after determining that the component data is the abnormal data, wherein the plurality of operations comprises:
in response to determining that the component data is the abnormal data, acquiring a sampling time of the abnormal data, and acquiring position information of the sensor for collecting the abnormal data; and
analyzing the N-component data in a time period during which the sampling time is located.

8. (canceled)

9. A system comprising:
a memory; and
a processor operatively coupled to the memory, the processor to:
acquire air quality data of a monitored area, wherein the air quality data comprises ground air quality data corresponding to the monitored area and N-component sample data collected by an air sensor in the monitored area, wherein N is a positive integer;
input the air quality data into a pollution analysis model, wherein the pollution analysis model is previously trained according to the ground air quality data and the N-component data; and
determine whether the air quality data of the monitored area is abnormal according to an output result of the pollution analysis model.

10. The system of claim 9, wherein the processor is further to train the pollution analysis model according to air quality training data by:
collecting the ground air quality sample data corresponding to the monitored area and the N-component sample data corresponding to a sample area at a preset sampling time interval;
determining an air quality label of the sample area according to the air quality sample data; and
constructing an air quality data set with the air quality label and the N-component sample data according to sampling time.

11. The system of claim 10, wherein, to determine the air quality label of the sample area according to the air quality sample data, the processor is further to:
acquire an aerosol optical thickness and trace gas quantitative remote sensing parameters in the air quality sample data; and
quantitatively determine the air quality label according to the aerosol optical thickness and trace gas quantitative remote sensing parameters.

12. The system of claim 10, wherein, to construct the air quality data set with the air quality label and the N-component sample data according to the sampling time, the processor is further to:
perform dimensionality reduction on the N-component sample data corresponding to the air quality label according to a principal component analysis method to obtain the air quality data set.

13. The system of claim 12, wherein the processor is further to: after constructing the air quality data set with the air quality label and the N-component sample data according to the sampling time, perform a plurality of operations, wherein the plurality of operations comprises:
dividing the air quality data set collected within a preset time period into training samples and test samples;
testing model parameters in the pollution analysis model according to the training samples; and
verifying whether the pollution analysis model is accurate according to the test samples.

14. The system of claim 9, wherein, to determine whether the air quality data of the monitored area is abnormal according to the output result of the pollution analysis model, the processor is further to:
in response to determining that the air quality data of the monitored area is abnormal, respectively compare a sampling duration in the N-component data with a corresponding preset abnormality duration threshold; and
in response to determining that the sampling duration of the component data is greater than the preset abnormality duration threshold, determine that the component data is abnormal data.

15. The system of claim 14, wherein the processor is further to perform a plurality of operations after determining that the component data is the abnormal data, wherein the plurality of operations comprises:
in response to determining that the component data is the abnormal data, acquiring a sampling time of the abnormal data, and acquiring position information of the sensor for collecting the abnormal data; and
analyzing the N-component data in a time period during which the sampling time is located.

16. A non-transitory machine-readable storage medium including instructions that, when accessed by a processor, cause the processor to:
acquire air quality data of a monitored area, wherein the air quality data comprises ground air quality data corresponding to the monitored area and N-component sample data collected by an air sensor in the monitored area, wherein N is a positive integer;
input the air quality data into a pollution analysis model, wherein the pollution analysis model is previously trained according to the ground air quality data and the N-component data; and
determine whether the air quality data of the monitored area is abnormal according to an output result of the pollution analysis model.

17. The non-transitory machine-readable storage medium of claim 16, wherein the processor is further to train the pollution analysis model according to air quality training data by:
collecting the ground air quality sample data corresponding to the monitored area and the N-component sample data corresponding to a sample area at a preset sampling time interval;
determining an air quality label of the sample area according to the air quality sample data; and
constructing an air quality data set with the air quality label and the N-component sample data according to sampling time.

18. The non-transitory machine-readable storage medium of claim 17, wherein, to determine the air quality label of the sample area according to the air quality sample data, the processor is further to:
acquire an aerosol optical thickness and trace gas quantitative remote sensing parameters in the air quality sample data; and
quantitatively determine the air quality label according to the aerosol optical thickness and trace gas quantitative remote sensing parameters.

19. The non-transitory machine-readable storage medium of claim 16, wherein, to construct the air quality data set with the air quality label and the N-component sample data according to the sampling time, the processor is further to:
perform dimensionality reduction on the N-component sample data corresponding to the air quality label according to a principal component analysis method to obtain the air quality data set.

20. The non-transitory machine-readable storage medium of claim 19, wherein the processor is further to: after constructing the air quality data set with the air quality label and the N-component sample data according to the sampling time, perform a plurality of operations, wherein the plurality of operations comprises:
dividing the air quality data set collected within a preset time period into training samples and test samples;
testing model parameters in the pollution analysis model according to the training samples; and
verifying whether the pollution analysis model is accurate according to the test samples.
